SEQUENCE LISTING (6 sequences)

SEQ ID NO: 1 - protein, 21 aa, Homo sapiens
  MOD_RES (3)..(3): Xaa is hydroxyproline
GPXGPXGPXGPXGPXGPXGPX

SEQ ID NO: 2 - protein, 15 aa, Homo sapiens
TRYLRIHPQSWVHQI

SEQ ID NO: 3 - protein, 5 aa, Homo sapiens
GRGDS

SEQ ID NO: 4 - protein, 8 aa, Homo sapiens
YMESRADR

SEQ ID NO: 5 - protein, 17 aa, Homo sapiens
  MOD_RES (1)..(1): ACETYLATION
RMAPEEEMDRPFLYVVR

SEQ ID NO: 6 - protein, 7 aa, Rana spp.
  MISC_FEATURE (7)..(7): The terminal K residue may be deleted
RVRSFYK

(19) United States
(12) Patent Application Publication
(10) Pub. No.: US 2016/0346327 A1
(43) Pub. Date: Dec. 1, 2016

(54) Synthetic Platelets

(71) Applicant: THE REGENTS OF THE UNIVERSITY OF CALIFORNIA, Oakland, CA (US)

(72) Inventors: Samir MITRAGOTRI, Santa Barbara, CA (US); Stefano MENEGATTI, Goleta, CA (US); Aaron ANSELMO, Cambridge, MA (US)

(73) Assignee: The Regents of the University of California

(21) Appl. No.: 15/116,178

(22) PCT Filed: Feb. 3, 2015
(86) PCT No.: PCT/US2015/014326

§ 371 Date: Aug. 2, 2016

Related U.S. Application Data
(60) Provisional application No. 61/935,297, filed on Feb. 3, 2014.

(51) Int. Cl. A61K 35/18; A61K 38/48; A61K 31/727; A61K 47/48; A61K 9/14

(52) U.S. Cl. CPC A61K 35/18; A61K 47/48215; A61K 9/146; A61K 31/727; A61K 38/482; C12Y 304/21068

(57) ABSTRACT

Provided herein are various functionalized particles comprising a shell, dendritic linkers, and functional moieties. The dendrimer linkers allow very large numbers of functional moieties to be bound to the shell. The functional moieties may comprise peptides which synergistically promote platelet aggregation and hemostasis in wounded tissues. The functionalized particles may further be effectors of wound healing, thrombolysis and other functions, depending on the selection of functional moiety. Functionalized polymers having these functions are provided as well.

### CROSS-REFERENCE TO RELATED APPLICATIONS

This application claims the benefit of priority to U.S. Provisional Application Ser. No. 61/935,297, entitled “Artificial Platelets and Related Systems,” filed Feb. 3, 2014, the contents which are hereby incorporated by reference.

### STATEMENT REGARDING FEDERALLY SPONSORED RESEARCH OR DEVELOPMENT

This invention was made with United States government support under Grant Number DGE-1144085 awarded by the National Science Foundation. The United States government has certain rights in the invention.

### REFERENCE TO SEQUENCE LISTING, A TABLE, OR A COMPUTER PROGRAM LISTING COMPACT DISK APPENDIX

This application is submitted with a computer readable sequence listing, submitted herewith via EFS as the ASCII text file named: “UCSB011PCT_ST25.txt,” file size approximately 4,096 bytes, created on Feb. 1, 2015 and hereby incorporated by reference in its entirety.

### BACKGROUND AND SUMMARY OF THE INVENTION

Excessive blood loss is responsible for approximately 3 million deaths worldwide due to trauma, and is the leading cause of preventable deaths following serious injuries. Hemostatic agents have the potential to prevent or reduce blood loss after serious wounds. In practice, however, hemostatic agents are often not effective in this task. For severe wounds, current emergency hemostats are usually administered externally as a hemostat-containing-gauze. While effective in the treatment of externally accessible wounds, these agents are unable to treat internal wounds, especially those which may have multiple bleeding sites. Application of current hemostats is further limited since the precise site of hemorrhage is not always known. Accordingly, there is a need in the art for hemostatic agents which can effectively overcome the shortcomings of the prior art.

Disclosed herein are novel functionalized particles for the delivery of various agents to the bloodstream and/or tissues of an animal. In one implementation, the functionalized particles of the invention can act as synthetic platelets. Platelets are an important component of the wound response in animals. Wounding results in the exposure of collagen, thrombin, and von Willebrand factor to the blood and causes the activation of platelets. Activated platelets clump together and begin the clotting process which stops bleeding. Unfortunately, unlike red blood cells, platelets cannot be stored for long periods of time. Accordingly, there is a need in the art for platelet substitutes which can aid in treating patients with wounds, including internal wounds. Additionally, there is a need for effective and selective agents which can aid in the initiation of clotting in wounded tissues. As described herein, the synthetic platelets of the invention can fulfill this unmet need. Additional embodiments of the invention include wound healing particles, anti-thrombotic particles, thrombolytic particles, and modified red blood cells having platelet functions, as set forth herein.

### BRIEF DESCRIPTION OF THE DRAWINGS

FIG. 1 is a schematic overview of the synthesis process for synthetic platelets.

FIG. 2A is a schematic depiction of a wound site without synthetic platelets. FIG. 2B is a schematic depiction of a wound site in which synthetic platelets are aiding in thrombus formation.

FIG. 3A depicts the synthesis of synthetic platelets. FIG. 3B depicts the activation of dendrimer COOH groups by CDI. FIG. 3C depicts the activation of dendrimer-peptide conjugates via EDC. FIG. 3D depicts coupling of the peptide on the activated dendrimer. FIG. 3E depicts artificial platelet activation via CDI.

### DETAILED DESCRIPTION OF THE INVENTION

Presented herein are novel compositions and methods encompassing functionalized particles, being termed “functionalized” because they carry selected biologically active moieties, which may be utilized in various medical applications.

In one aspect, the functionalized particles of the invention comprise synthetic platelets which may be used as thrombogenic agents. Alternative embodiments comprise thrombolytic agents, healing agents, hemostatic peptide-polymer conjugates, wound healing peptide-polymer conjugates, anti-thrombolytic peptide-polymer conjugates, thrombolytic polymer-peptide conjugates and other agents. In another aspect, the invention comprises methods and compositions encompassing the modification of cells and cell components to create agents with platelet functions.

Functionalized Particles.

The functionalized particles of the invention, in general, comprise three elements: (1) a substrate; (2) linkers; and (3) functional moieties. Linkers are anchored on the outer surface of the substrate. Functional moieties are bonded to the terminal ends of the linkers. The size, shape, and composition of the substrate, the linker properties, and the identity of the functional moieties will be selected depending on the desired functionality of the particles.

In one implementation, the substrate comprises a shell formed over a core, the core optionally being dissolved or degraded subsequent to shelf formation, leaving the shell hollow. The choice of core shape, size and composition, along with the shell composition and thickness, will determine the final properties of the shell.

The Core.

In those embodiments utilizing a shell, the function of the core is to provide a substrate for the creation of the shell. The core will define the shape and the size of the functional body. In some embodiments, the core is retained and makes up part of the final product. In other embodiments, subsequent to formation of the shell, the core is partially or wholly degraded and the resulting shell is substantially hollow. The core may be comprised of any material or materials, so long as the outer surface of the core will support synthesis, deposition, or formation of the shell thereon. For example, a core having an outer surface capable of binding cationic/anionic polymers, polyelectrolytes, or proteins may be used.

Exemplary core materials include polystyrene (PS), poly (lactic-co-glycolic) acid (PAH), Hyaluronic acid (HA), calcium hydroxide (CaOH₂), and silica materials. Core materials that can be readily dissolved or digested are used in those embodiments where a hollow shell is desired. Materials amenable to digestion by polar solvents, aqueous solvents, acids, bases, enzymes, etc may be used.

Optionally, materials may be embedded or encased within the core, such that when the core is dissolved, these materials remain within the shell. Exemplary materials for inclusion in the core include drugs, for example encapsulated in slow-release dissolving materials), markers (e.g. fluorophores, quantum dots, or other visible markers), gene therapy constructs (e.g. nucleic acids, optionally encapsulated), and other materials.

The size and of the core will largely determine the final size and shape of the shell deposited onto it. Core size may vary from a few nanometers to over ten micrometers. For intravenous uses, bodies larger than 10 micrometers may cause cardiopulmonary complications, for example by aggregating in capillaries. For intravenous uses, core sizes in the range of 10-500 nanometers are effective for efficient circulation in the body.

The shape of the core may vary. In general, cores that are substantially spherical or elliptical may be used.

The Shell.

The shell comprises a polymeric, proteinaceous, or other material which is synthesized, deposited, or otherwise formed on the outer surface of the core. Exemplary shell materials include proteins, polyelectrolytes, and polymers. The shell materials will generally be biocompatible. The shell materials will optionally be somewhat flexible. The shell materials are not limited to permeable materials. For example, in embodiments where retaining the core is advantageous, the shell materials may be substantially impermeable. In those embodiments where the core is to be partially or wholly dissolved or digested, the shell materials must be adequately porous or permeable to the dissolution agent that it can reach the core, and must be sufficiently inert or resistant to the dissolution agent that the shell will not be significantly degraded when removing the core from the body.

In one embodiment, the shell material comprises a multilayer structure formed using layer-by-layer synthesis. Such structures are formed by the sequential layering of two materials, the two materials typically having opposite charges in order to facilitate adsorption of each layer on the other. Advantageously, layer-by-layer synthesis allows a high degree of control over shell thickness. After the alternating bi-layers have been deposited, a cross-linking or other fixative step is performed to bond and strengthen the shell. For example, chemical cross linking, UV-activated cross linking, and/or the inclusion of intercalated cross-linking agents may be used. Any number of bilayers may be used, for example 1 to 20, depending on the desired qualities of the finished shell. For example, if a very thin and flexible shell is desired, a low number (e.g. 1 or 2) of bilayers may be used. When small numbers of bilayers are used, a more substantial crosslinking process is required to ensure the strength of the shell. Thicker shells can be created using higher numbers of bi-layers.

Various layer-by-layer synthesis methods are known in the art, for example as described in: Wang, Y., A. S. Angelatos, and F. Caruso, Template Synthesis of Nanostructured Materials via Layer-by-layer Assembly. Chemistry of Materials, 2007. 20(3): p. 848-858; Zhou, Z., A. C. Anselmo, and S. Mitragotri, Synthesis of protein-based, rod-shaped particles from spherical templates using layer-by-layer assembly. Adv Mater, 2013. 25(19): p. 2723-7; Doshi, N., et al., Platelet mimetic particles for targeting thrombi in flowing blood. Adv Mater, 2012. 24(28): p. 3864-9; Doshi, N., et al., Red blood cell-mimicking synthetic biomaterial particles. Proc Natl Acad Sci USA, 2009. 106(51): p. 21495-9; del Mercato, L. L., et al., LbL multilayer capsules: recent progress and future outlook for their use in life sciences. Nanoscale, 2010. 2(4): p. 458-67; Johnston, A. P., et al., Layer-by-layer engineered capsules and their applications. Current Opinion in Colloid & Interface Science, 2006. 11(4): p. 203-209; and Yan, Y., M. Björnmalm, and F. Caruso, Assembly of Layer-by-Layer Particles and Their Interactions with Biological Systems. Chemistry of Materials, 2013.

An exemplary system for shell formation is the use of alternating bi-layers of poly (allylamine hydrochrolide) (PAH) and bovine serum albumin (BSA). Alternatively, actin and PAH layers may be used. Additional exemplary shell materials include Poly-L-lysine, Actin, Hemoglobin, human serum albumin, poly(4-styrene sulfonate), PMA, PVPON, Chitosan, dextran, and alginate, as known in the art. Further exemplary shell materials include any protein pair, wherein one protein has an isoelectric point greater than 7, and the second protein has an isoelectric point less than 7; a positively charged synthetic polymer and a negatively charged polymer; and other self assembling molecules.

Core Dissolution.

After the shell layer has been synthesized, deposited, or formed, the core may optionally be dissolved, degraded, or otherwise removed. In one embodiment, the dissolution is effected by exposing the shell and core to a solution which dissolves the core material but which does not significantly affect the shell material(s). For example, when a polystyrene core has been utilized, it can be dissolved by exposure to a tetrahydrofuran-isopropyl alcohol gradient, as described in Example 1. The choice of appropriate dissolution agent will depend on the composition of the core and the shell.

Upon removal of the core, depending on the material comprising the shell, the remaining shell will be flexible. For example, BSA-PAH shells will exhibit high flexibility, which aids in in vivo circulation of the particles. When a substantially spherical core is used, upon core dissolution, a flexible shell such as BSA-PAH will assume a discoid platelet-like structure.

Functionalized Polymers.

Another implementation of the invention encompasses the use of polymers, as opposed to proteins, as the functional agent-harboring scaffold or substrate. Likewise, hybrid substrates comprising bilayers of protein and polymeric material may be employed as the substrate. It will be understood that the methods of functionalizing shells and utilizing functionalized shells described herein are equally applicable to functionalized particles wherein a polymeric material not configured as a shell replaces the shell as the substrate for functional moieties. Examples of polymers that could be used include hyaluronic acid, polyvinyl alcohol, or polylactic-co-glycolic acid. As with the shell, linkers are anchored on the polymerchain, the linkers having functional moieties are bonded to the terminal ends of the linkers.

The choice of polymer type, molecular weight, composition, linker properties, and the identity of the functional moieties will be selected depending on the desired functionality of the polymer conjugates. Polymeric materials may be formed into any number of structures, including shells around a core (wherein the core may optionally be dissolved), discoid or spherical bodies, planar bodies, fibers, and other secondary structures. In one embodiment, the polymeric material comprises a single polymer chain.

Linkers.

In one embodiment, the functional moieties may be directly attached to the substrate (e.g. the shell or polymeric substrate) without the use of any linker moiety. Alternatively, in many implementations, the functional moieties are attached to the substrate using linkers. A linker is any molecule capable of (1) binding to the substrate material, and (2) which is also capable of binding a functional agent, typically at its terminal end(s). Linkers anchor the functional moiety to the substrate, and in some cases serve the role of spacer, holding the functional moiety a distance off the surface of the substrate to avoid interactions between the functional moiety and the substrate material. For example, linkers of 2-3 times the length of the functional moiety may be used to hold such compound at a distance from the substrate surface that will avoid undesired interactions.

Where functional moieties of highly divergent sizes are used, the smaller moieties can be placed at the ends of longer linkers which compensate for the size difference between the moieties, such that both are displayed at about the same plane the functional particle's outer perimeter. The smaller species may be tethered to a spacer molecule, for example a PEG molecule, the length of which is selected to match the size of the larger species (e.g. PEG linkers ranging from 500 to 5,000 kD).

Further, linkers can control the density of functional moieties and can control the display geometry of the functional moieties, for example separating portions of the substrate surface displaying a selected functional moiety from other portions displaying different moieties.

In one embodiment, the linkers comprise highly branched molecules, for example dendrimers. The advantage of using a highly branched molecule as a linker is that a single anchoring site on the substrate surface can serve as attachment point for numerous functional moieties, greatly increasing the effective concentration of functional moieties, and thus the binding avidity of the body to the biological target. Exemplary branched linkers include dendrimers, for example dendrimers having anywhere from 2 to 2,000 branches. For example, a dendrimer having 44 to 2,048 or more branches can be utilized, e.g. dendrimers of generation 2-10 or more. Higher generation dendrimers increase the surface area of the functional body and the number of active molecules. Exemplary biocompatible dendrimers include poly(amidoamine) (“PAMAM”) dendrimers and peptide-based dendrimers, as known in the art.

Functional Moieties.

The functional bodies of the invention comprise one or more types of functional agents linked to the susbstrate by the linker. The functional moieties may comprise any material which imparts properties or functions to the functional body. Properties and functions include binding ability and avidity for target proteins or cell types, enzymatic activity, and any other biological ability, effect, or action. Functional moieties may include, for example, proteins, peptides, peptidomimetics, antibodies, enzymes, binding sites, markers, fluorescent probes, affinity tags, chelating agents, radioactive probes, etc.

Multiple types of functional moieties may be included in the functionalized particle. For example, in the artificial platelet implementation of the invention described below, three different peptides are included to impart the desired thrombogenic function. Any number of desired compounds may be included. In one embodiment, in addition to specific functional moieties that impart biological function, ancillary moieties are included which aid in the circulation of the functional body in the bloodstream of the target animal and the retention of the functional body within a living organism, such as moieties which increase solubility or reduce immunogenicity.

When multiple types of functional moiety are to be included in a functionalized particle, it will generally be advantageous to perform separate conjugation reactions between each type of functional moiety and the linker Linker-functional moiety conjugates can then be joined to the substrate in a single conjugation reaction, wherein the different types of linker-functional moiety conjugates are present in the stochastic ratios desired for the final product. Alternatively, different linker-functional moiety species can be added to substrate in a serial fashion to control relative densities. Alternatively, linkers can be first conjugated to the substrate, followed by functionalization reactions to join functional moieties to the linkers

Conjugation of functional moieties to linkers, and linkers to substrate materials can be accomplished using any conjugation chemistries known in the art which are compatible with the shell material, linker composition, and makeup of the functional moiety. For example, PAMAM dendrimers with terminal COOH— groups can be activated with carbonyldiimidazole and subsequently linked with peptide amino groups to conjugate linkers to functional moieties comprising peptides or other compounds having a terminal amino group. Likewise, EDC/NHS coupling, as known in the art, can be used to link dendrimer-peptide conjugates to the surface of a proteinaceous shell. In general, any carbodiimide chemistry can be used to conjugate linkers to functional moieties and to shell materials. Other chemistries that could be employed include activation of the carboxyl group on the dendrimer with any agent that forms a carboxyl chloride, or an activated ester, to react with the nucleophiles on the shell.

The density of functional moieties per body may vary considerably, from less than 50,000 per body to >100 million per body. For example 10-30 million functional moieties may be present on a shell of 200 nm in diameter. The density of functional moieties on the shell can be tuned by controlling the concentration of the dendrimer-peptide conjugates in the coupling solution during the reaction with the shell. Alternatively, by controlling the size, and thus the surface area, of the substrate used.

In one aspect, the invention broadly encompasses any functional particle comprising a substrate (e.g. a shell or polymeric material), linkers, and functional moieties, for example: wherein the shell is a layer-by-layer structure, for example a structure comprising one or more BSA-PAH bilayers, which such layer-by-layer structure is formed around a particulate core, the core optionally being degraded or dissolved subsequent to formation of the shell; wherein the linkers may comprise branched linkers, for example dendritic linkers such as PAMAM dendrimers; and wherein the one or more functional moieties comprise a biologically active or biologically targeting agent. It will be understood that variants of the above structure fall within the scope of the invention, for example the shell may comprise a structure other than a layer-by-layer structure (for example, the polymer-conjugates described above) or the linkers may be linear rather than branched.

For convenience, the systems described herein are directed to humans, including peptide sequences which are derived from or are otherwise biocompatible with humans. The invention further encompasses functionalized particles comprising materials that may be used in canines, felines, rats, mice, cows, pigs, monkeys, and other species, including appropriate homologs or orthologs of the human sequences or sequences described herein.

The invention further encompasses methods of utilizing the functionalized particles or polymers described herein. In one embodiment, the invention comprises the administration of a functionalized particle to an animal in need of treatment, the functional moieties of the particle being effectors of the required treatment. Such administration may be intravenous, topical, or may comprise a localized injection or other delivery. It will be understood that the functionalized particles or polymers may be administered in or with pharmaceutically acceptable carriers, including for example, solutions, gels, or particulates. The invention further encompasses kits, wherein such kits may comprise: functionalized particles in combination with pharmaceutically acceptable carriers; functionalized particles in combination with adjunct or accessory agents (e.g. drugs); and functionalized particles in combination with delivery mechanisms, such as syringes, hypodermic needles, and intravenous needles. The invention includes the use of the compositions described herein in bandages, dressings, sutures, and other wound treatment articles. The invention further encompasses methods of delivering functionalized particles to cells, including isolated cells, tissue explants, cultured cells, and others. The methods of the invention encompass medical therapeutic treatment of humans, veterinary treatments, and research uses.

Various embodiments of the invention are described next.

Synthetic Platelets for Promoting Platelet Aggregation.

In one embodiment, the invention comprises synthetic platelets that promote thrombus formation in wounded tissues. The thrombogenic synthetic platelets of the invention comprise the general functionalized particle configurations described above, wherein the one or more functional moieties comprise a wound-targeting ligand and a thrombogenic agent, for example a platelet aggregation agonist or platelet binding agent. The wound-targeting ligand comprises any agent which selectively binds to peptides or other species which are presented by damaged endothelium cells. For example, in one embodiment, the wound-targeting ligand comprise a collagen binding agent. In another embodiment, the wound-targeting ligand comprises a von Willebrand binding agent. In another embodiment, the platelet aggregation agonist or platelet binding agent comprises a fibrinogen mimetic. In one implementation the platelet aggregation promoting particles comprise three functional moieties: a collagen-binding peptide (CBP); a von Willebrand binding peptide (VBP); and a fibrinogen mimetic peptide (FMP). Typically, these functional moieties will comprise peptides. These three functional moieties act synergistically to effectively promote thrombus formation. Specifically, the CBP and VBP moieties promote adhesion to fibrinogen while the FMP moiety enhances cross-binding to native platelets, aiding in thrombus formation.

The CBD moiety of the synthetic platelets of the invention comprises any collagen-binding peptide or other collagen-binding agent known in the art. The task of this moiety is to achieve adhesion of the artificial platelet to wounded tissues (where collagen has become exposed to the blood) at low blood flow rates. For example, in one embodiment, the CBD moiety of the invention comprises SEQ ID NO: 1, known as [GPO]₇, a seven-mer of the tripeptide glycine-proline-hydroxyproline. For example, see Kehrel B., Wierwille S., Clemetson K. J., Anders O., Steiner M., Knight C. G., Farndale R. W., Okuma M., Barnes M. J. “Glycoprotein VI is a major collagen receptor for platelet activation: it recognizes the platelet-activating quaternary structure of collagen, whereas CD36, glycoprotein IIb/IIIa, and von Willebrand factor do not.” Blood 1998; 91: 491-9; and Farndale R. W., Sixma J. J., Barnes M. J., de Groot P. G., The role of collagen in thrombosis and haemostasis. J Thromb Haemost 2004; 2: 561-73.

Other exemplary CBD peptides include, for example, sequences described in Munnix et al., 2008, “Collagen-mimetic peptides mediate flow-dependent thrombus formation by high- or low-affinity binding of integrin a2b1 and glycoprotein VI,” Journal of Thrombosis and Haemostasis, 6: 2132-2142.

The von Willebrand binding peptide, VBP, of the synthetic platelets of the invention comprises any peptide or other agent that effectively binds von Willebrand factor. The task of this moiety is to achieve adhesion of the artificial platelet to wounded tissues, where von Willebrand factor has become exposed to the blood, at high blood flow rates. In one embodiment, the VBP may comprise a peptide having SEQ ID NO: 2, which is a human-derived sequence, abstracted from Factor VIII, which is a natural protein ligand for the vWF. Other exemplary VBP's include: those described in published PCT patent application number WO 2007052067, entitled “Von willebrand factor (vwf) binding peptides,” by Farndale et al.; sequences described in Moriki et al., 2010, “Identification of ADAMTS13 Peptide Sequences Binding to Von Willebrand Factor,” Biochemistry Biophys Res Commun, 391:783-788; and sequences described in Lisman et al., 2006, “A single high-affinity binding site for collagen on von Willebrand factor in collagen III, identified using synthetic triple helical peptides,” Blood 108: 3753-56. VBP may further include peptides described in Huang, et al., “Affinity purification of von Willebrand factor using ligands derived from peptide libraries,” Bioorg Med Chem. 1996 May; 4(5):699-708, for example SEQ ID NO: NO 6.

The thrombogenic moiety of the synthetic platelets of the invention comprises any agent which promotes thrombogenesis at wound sites (while avoiding off-target thrombus formation). The thrombogenic moiety may comprise a fibrinogen mimetic peptide, or any other agent which effectively binds the GPIIb/IIIa site of activated platelets, which aids in crosslinking activated platelets and promotes platelet aggregation at the wound site. In one embodiment, the FMP comprises SEQ ID NO: 3. In another embodiment, the FMP moiety of the synthetic platelets of the invention may comprise SEQ ID NO: 4, which is less likely to induce off-target platelet activation than SEQ ID NO: 3. Any RGD-based fibrinogen peptide peptide may be used. Non-peptide binders of fibrinogen may also be employed, for example as described in Sugihara, et al., “Novel Non-Peptide Fibrinogen Receptor Antagonists. 1. Synthesis and Glycoprotein IIb-IIIa Antagonistic Activities of 1,3,4-Trisubstituted 2-Oxopiperazine Derivatives Incorporating Side-Chain Functions of the RGDF Peptide,” J. Med. Chem., 1998, 41 (4), pp 489-502.

With respect to the specific functional moieties described for the synthetic platelets, and for all other embodiments of the functionalized particles and modified cells described below, it is understood that one of skill in the art may readily select and utilize functional analogs of the peptides disclosed herein in place of the described peptides. For example, any molecule, including for example small molecules, peptides, proteins or fragments thereof, polymers, antibodies, modified sequences (including sequences comprising non-natural amino acids) or any other composition of matter capable of effecting the same or similar physiological response may be used in place of the exemplary peptides set forth herein. Likewise, one of skill in the art may select variants of SEQ ID NO: 1, SEQ ID NO: 2, SEQ ID NO: 3, SEQ ID NO: 4, SEQ ID NO: 5 and other sequences described herein. As used herein, a variant of a peptide is a sequence having one or more amino acid substitutions and which retains some or all of the original peptide's function.

The relative proportions of CBP, VBP, and FMP in the synthetic platelets of the invention may vary. For example, CBP:VBP:FMP ratios of 1:1:1, 1:1:2, or 1:1:3 may be utilized. It will be understood that in some alternative embodiments, one binding moiety, e.g. CMB or VBP, may be omitted, however, the use of both enhances particle effectiveness.

The synthetic platelets described herein may be utilized in various ways. For example, in one embodiment, the invention comprises a method wherein synthetic platelets are injected into the blood stream of an animal, e.g. a human patient, in need of treatment, e.g. having a wound or otherwise needing hemostatic treatment, for example, in order to enhance thrombus formation at externally accessible or internally bleeding sites. For intravenous injection, doses in the range of 0.0001%-500% of the normal platelet concentration of the treated animal species may be utilized. For example, synthetic platelet does of 3-75 mg/kg, for example 15 mg/kg may be used. In an alternative method, the synthetic platelets are applied topically to wounds or surgical incisions of an animal, e.g. a human patient and may be incorporated into bandages, dressings, etc.

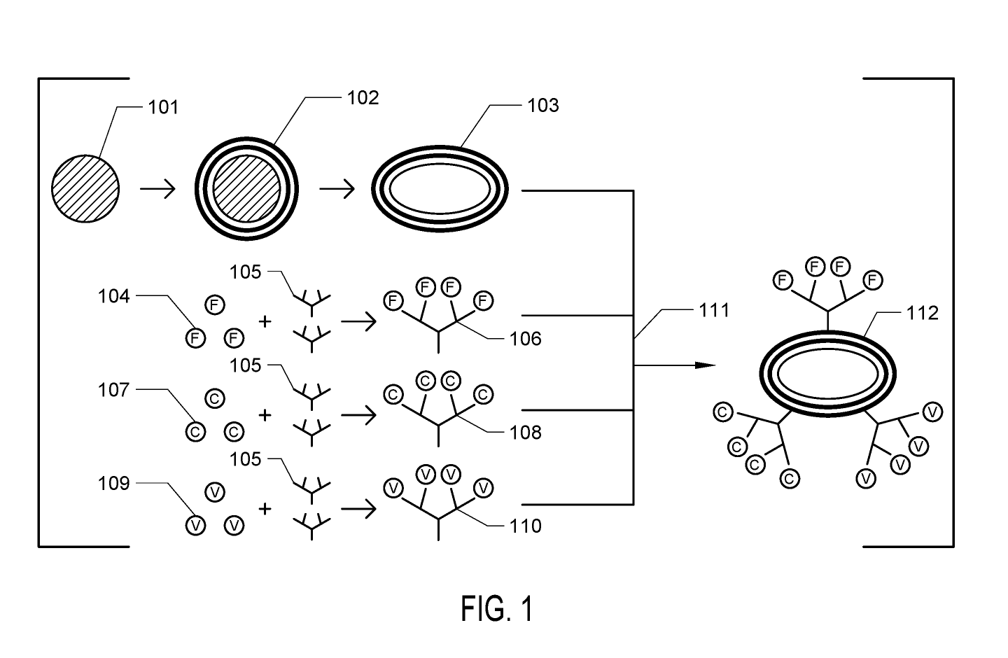

An exemplary synthesis of the artificial hemostatic platelets of the invention is depicted schematically in FIG. 1. Here, a core 101 is coated with a polymer shell 102. After the shell is synthesized, the core is dissolved, leaving a hollow, flexible, discoid body formed by the shell 103. In a first reaction, fibrinogen mimetic peptides 104 are conjugated with dendrimers 105, yielding dendrimer-conjugated fibrinogen mimetic peptides 106. In a separate reaction, collagen binding peptides 107 are conjugated with dendrimers 105, yielding dendrimer-conjugated collagen-binding peptides 108. In a third separate reaction, von Willebrand-binding peptides 109 are conjugated with dendrimers 105, yielding dendrimer conjugated von Willebrand-binding peptides 110. In a single reaction 111, the three batches of dendrimer-conjugated peptides are reacted with activated shells, yielding shells that are decorated with dendrimers conjugated to the three functional peptides 112.

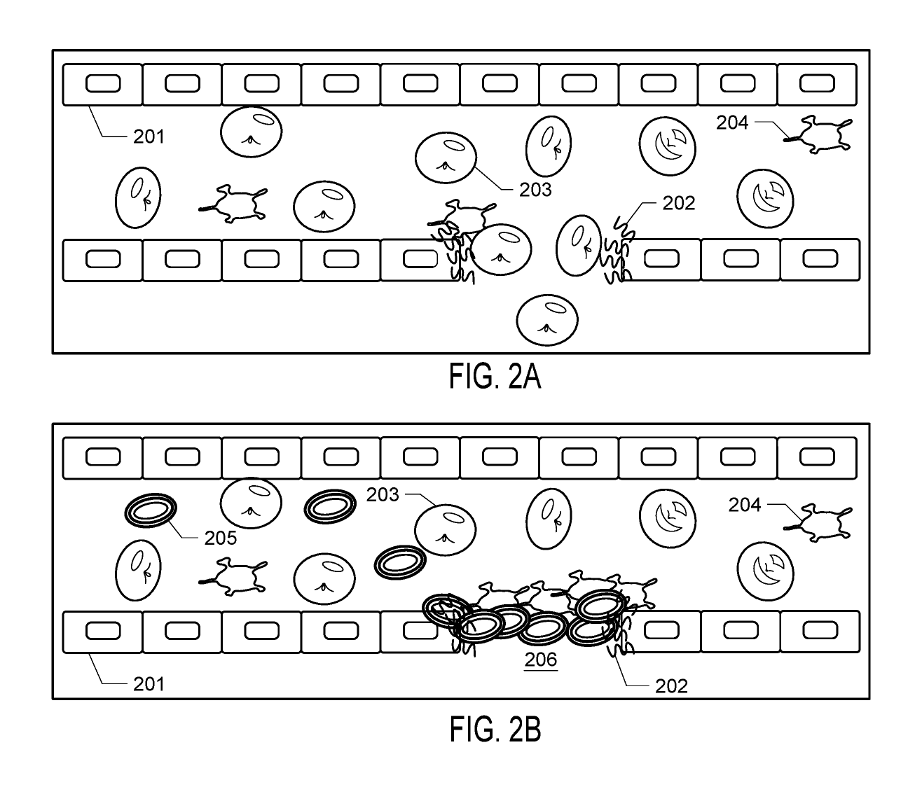

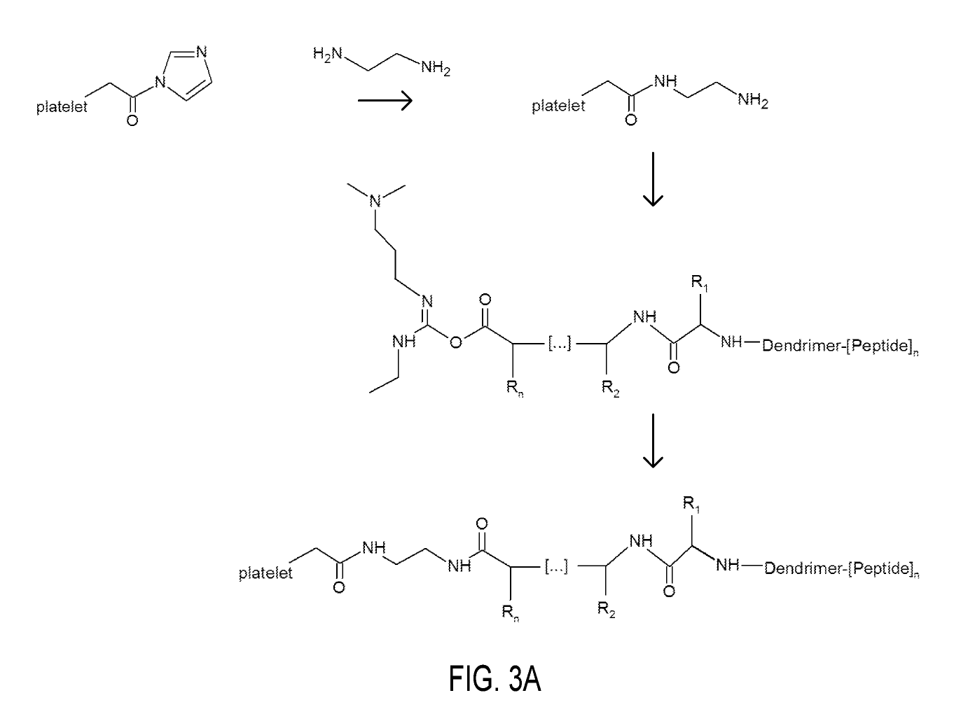

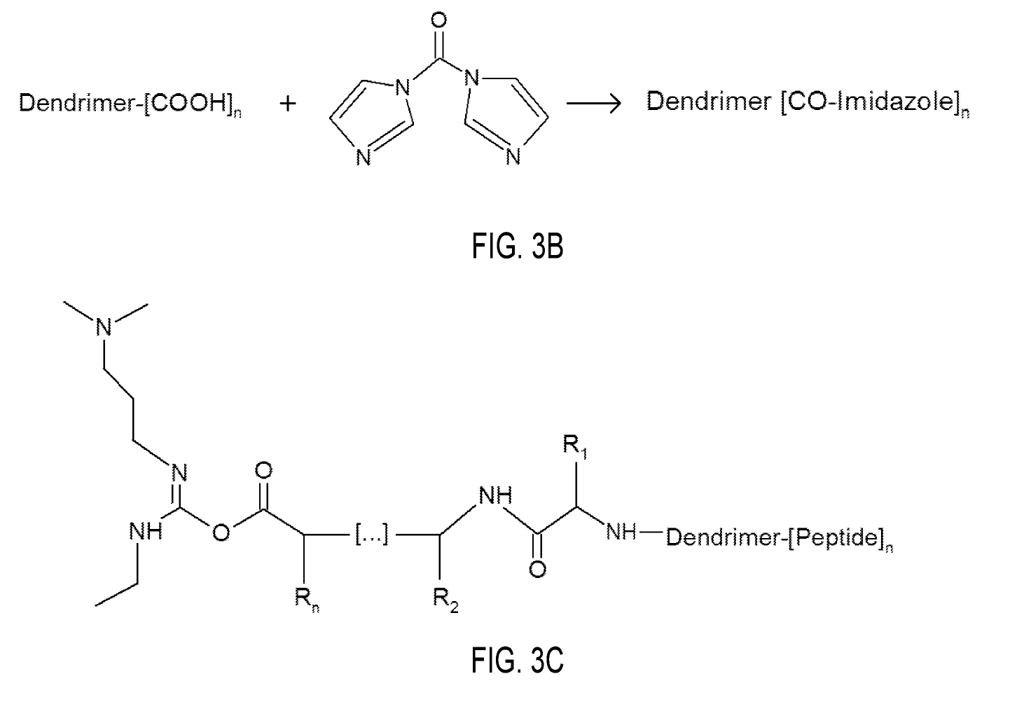

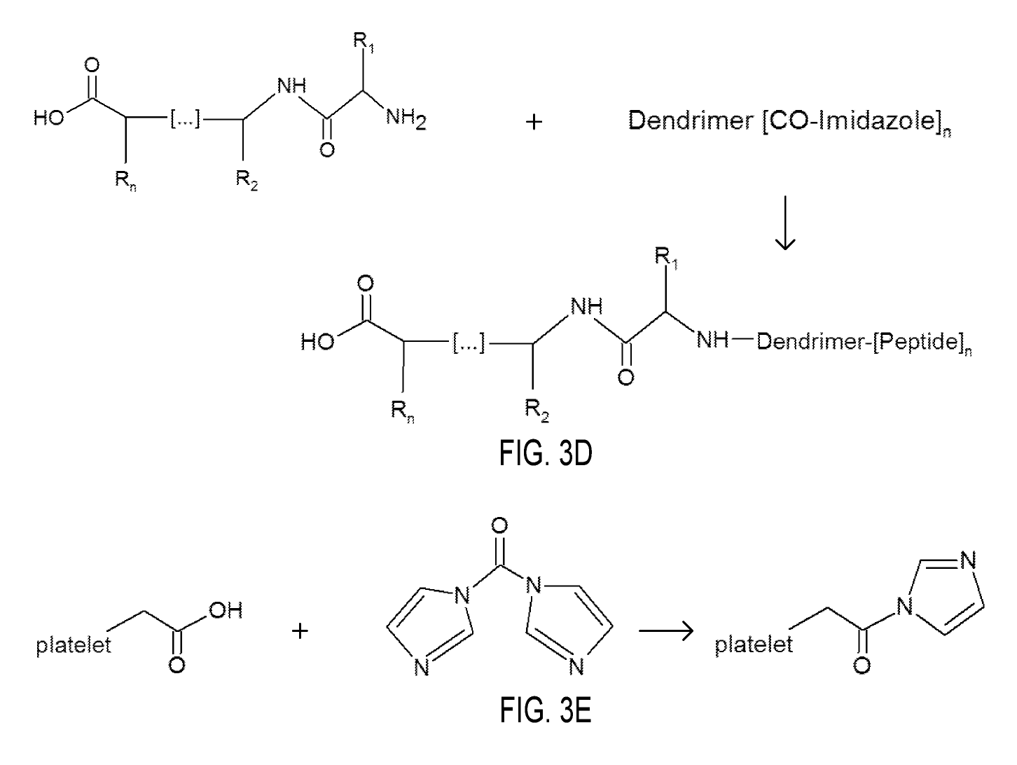

With reference to FIG. 2A and FIG. 2B, these schematic diagrams depict wound sites. In FIG. 2A, healthy endothelial cells 101 surround a wound site, where wound-specific factors 102 are exposed. Red blood cells 103 are escaping from the wound site, as platelets 104 are not abundant enough to form a thrombus. In FIG. 2B, the synthetic platelets of the invention 205 are present, and a thrombus 206 is rapidly formed to seal the wound.

Wound Healing Particles.

Subsequent to clot formation, wound healing processes repair the damaged tissue. Various wound healing peptides (WHP's) are known to promote the healing process. Histatin or derivatives thereof, for example, may act as a wound healing peptide. For example, WHP's include those described in Demidova-Rice et al., “Human Platelet-Rich Plasma- and Extracellular Matrix-Derived Peptides Promote Impaired Cutaneous Wound Healing In Vivo,” 2012, PLOS ONE, DOI: 10.1371/journal.pone.0032146; Demidova-Rice et al., “Bioactive peptides derived from vascular endothelial cell extracellular matrices promote microvascular morphogenesis and wound healing in vitro,” Wound Repair Regen. 2011 19(1):59-70; United States Published Patent Application number 2013010861, entitled “Wound Healing Peptides and Uses Thereof,” by Herman et al.; and frog skin-derived peptides described in Liu et al., “A potential wound healing-promoting peptide from frog skin,” Int J Biochem Cell Biol. 2014 April; 49:32-41, SEQ ID NO: 6.

In one embodiment, the invention comprises wound healing particles. The wound healing particles comprise the basic functionalized particle configurations described above, i.e. a shell and linkers having terminal functional moieties, and further comprise: (1) a wound-targeting ligand; and (2) a wound healing agent. The wound-targeting ligand may be any agent which selectively binds to species that are present in wounded tissues, e.g. collagen, von Willebrand factor, etc. The wound healing agent may be any agent that promotes healing of wounds.

In the wound healing particles, the wound binding moieties may comprise CBP, VBP, and other compounds known in the art to selectively bind to wound specific sites, motifs, cells, etc. For example, arginylglycylaspartic acid (RGD) and peptides based thereon may be used. The wound healing agent may comprise those listed above or any other known in the art. The wound binding moieties, e.g. CBP and VBP, target the wound healing particles to wound sites, wherein the WHP's can promote healing processes at the wound site.

The invention further comprises methods of administering such wound healing particles to an animal in need of treatment, e.g. a human patient having one or more wounds or wounded tissues. Wound healing particle doses may be administered at any physiologically effective dose which does not cause excessive adverse side effects, for example, doses of 3-75 mg/kg, for example 15 mg/kg may be used.

Anti-Thrombotic or Thrombolytic Particle.

Pathological thrombosis is implicated in many conditions. To treat such conditions, anti-thrombosis or thrombolytic compounds need to be delivered to the site of the undesired clotting activity or clot. In one embodiment, the invention comprises particles capable of binding to clotting sites and delivering anti-thrombotic or thrombolytic agents. The anti-thrombotic or thrombolytic particles of the invention comprise the basic functionalized particle configurations described above, i.e. a shell and linkers having terminal functional moieties.

The functional moieties of the anti-thrombotic particles include (1) a targeting agent that binds the particle to factors found in clotting sites, such as activated platelets, and (2) an anti-thrombotic agent. In one embodiment, SEQ ID NO: 4 may be used as the targeting entity which binds the particle to clot-specific or clot-associated factors at the site of the pathological event. Alternatively, SEQ ID NO: 3 may be used as well. In one embodiment, heparin is utilized as the anti-thrombotic agent. Other thrombolytic drugs or peptides known in the art may be used in combination with or in place of heparin.

The functional moieties of the thrombolytic particles include (1) a targeting agent that binds the particle to wounded tissue sites, and (2) thrombolytic agents that degrade clots or promote clot dissolution. In one embodiment, SEQ ID NO: 4 may be used as the targeting entity which binds the particle to clot-specific or clot associated factors at the site of the pathological event. In one embodiment, tPA is utilized as the thrombolytic moiety.

It will be noted that protein-based thrombolytic agents like tPA are very large molecules and can decrease the display of the smaller targeting peptide by steric crowding. It is possible to obviate to this problem by binding the thrombus targeting peptide onto a long PEG or other tethering moiety, the length being that which will place the targeting peptide at about the same plane as the large tPA or other large thrombolytic molecule. For example a PEG molecule of 500 to 5,000 kDa may be used.

In an alternative embodiment, the thrombolytic platelets of the invention comprise a tPA-recruiting particle, comprising a targeting functional moiety, such as SEQ ID NO: 4, and a tPA ligand, which such ligand is capable of binding tPA in an active configuration. An exemplary tPA ligand is the peptide SEQ ID NO: 5. Such tPA-recruiting particles can bind to the clot by means of the targeting moiety and then the tPA ligand will recruit native or co-administered tPA to that region, which in turn aids in clot dissolution.

In one aspect, the invention comprises methods of administering anti-thrombotic particles, thrombolytic particles, or tPA-recruiting particles to an animal in need of anti-thrombotic or thrombolytic treatment, e.g. a human patient, for example having undesirable clotting activity or clots. For example, the particles may be administered to an animal to aid in dissolving deep venous thrombi. Administration of the particles may be intravenous or topical. In one implementation, the invention comprises the administration of tPA-recruiting particles in combination with tPA. Anti-thrombosis, thrombolytic, and tPA-recruiting particles each may be administered at any physiologically effective dose which does not cause excessive adverse side effects, for example, doses of 3-75 mg/kg, for example 15 mg/kg, may be used.

Modified Red Blood Cells.

In a further aspect, the invention comprises red blood cells (or fragments thereof) which are modified to perform platelet functions. The modified red blood cells of the invention comprise red blood cells (or fragments thereof) which display agents having platelet functions, for example wound binding functions and thrombogenic agents such as platelet aggregation agonists or platelet binding agents. For example in one embodiment, the modified red blood cells of the invention display CBP, VBP, and FMP peptides tethered to their surface membrane. In this implementation of the invention, the red blood cells take the place of the shell as the substrate for functional moiety attachment. As with the synthetic platelets of the invention, the surface-bound CBP, VBP, and FMP will effect binding to wounded tissues and promotion of platelet aggregation. Such active moieties may be tethered to the outer membrane of the red blood cell using linear or branched linkers, for example PAMAM dendrimers. Such active moieties may be present in the same relative proportions described above for artificial thrombogenic platelets, for example, CBP:VBP:FMP ratios of 1:1:1 or 1:1:2. Any surface concentration of functional moieties, for example 50,000 to 100 million functional moieties per red blood cell, may be utilized.

Any methodology known in the art for surface modification of red blood cells may be used to attach the functional moieties to the red blood cell membrane, for example as described in: Henry S M, “Modification of red blood cells for laboratory quality control use,” Curr Opin Hematol. 2009 November; 16(6):467-72; U.S. Pat. No. 6,946,127, entitled “Decorated red blood cells,” by Bitensky et al.; and U.S. Pat. No. 8,211,656, entitled “Biological Targeting Compositions and Methods of Using the Same” by Hyde et al.

The invention further comprises methods of administering modified red blood cells having platelet functions to an animal, e.g. a human patient, in need of treatment, for example, in order to enhance thrombus formation at wounded, e.g. internally bleeding sites. Administration may be intravenous, topical, or may comprise a localized injection. Modified red blood cell may be administered at any physiologically effective dose which does not cause excessive adverse side effects, for example, doses of 3-75 mg/kg, for example 15 mg/kg may be used.

### Example 1

In this example, artificial thrombogenic platelets, referred to as SynPlats, were made and are tested in live animals.

Synthetic Nanoplatelets Fabrication.

200 nm carboxylate PS spheres (Polysciences, Warrington, Pa.) were suspended in 0.5 M sodium chloride (Fisher). 2 mg/ml of positively-charged Poly(allylamine) hydrochloride (Sigma) was dissolved in 0.5 M sodium chloride and incubated with 3×10¹² PS particles at room temperature under constant rotation for 30 minutes. Particles were then centrifuged at 15000 g for 30 minutes and resuspended in 0.5 M sodium chloride. Particles were washed 2 more times at 15000 g for 30 minutes in 0.5 M sodium chloride. Following PAH coating, negatively-charged bovine serum albumin (Sigma) was coated onto of PAH layers under identical conditions. This procedure was repeated for multiple (PAH/BSA) bilayers. Intermittent crosslinking with 2% glutaraldehyde (Polysciences) for 1 hour under constant rotation was performed to ensure sufficient structural integrity of the outer shells. The particles were then exposed to a tetrahydrofuran-isopropyl alcohol gradient (1:3, 1:2, 1:1, 2:1, and pure THF) for 30 minutes each at room temperature under constant rotation so as to dissolve the PS core. Particles were then washed 10× with saline (BD), so as to remove any residual solvent, sterilized via UV overnight and stored at 4 C for no longer than 2 days.

Peptide Conjugation to Synthetic Nanoplatelets.

In this example, CBP comprising SEQ ID NO: 1, VBP comprising SEQ ID NO: 2, and FMP comprising SEQ ID NO: 3 were utilized. Each of the three peptides were, in separate batches, coupled to poly(amido amine) (PAMAM) dendrimers (Sigma) via EDC/NHS chemistry in MES buffer at pH=4.5. Purification of dendrimer-peptide conjugates was performed via size exclusion chromatography. The outer layer of SynPlats were activated with cabonyldiimidazole (CDI) at 1 mg/ml in acetone for 45 minutes and diaminoethane was then added to form primary amino groups on SynPlat surface. All SynPlats were tested qualitatively for free amines via the Kaiser test. Finally, dendrimer-peptide conjugates were conjugated to SynPlats via EDC/NHS chemistry in MES buffer at 4.5 pH for 12 hours.

Scanning Electron Microscopy (SEM).

An FEI XL40 SEM at 3-10 kV with a 5 mm working distance was used for imaging particles. Samples were coated with palladium (at 10 kV) via a Hummer sputtering system.

In vivo hemostasis. 3×10¹⁰ SynPlats in saline were injected via tail vein into healthy female BALB/c mice (18-20 g; n=3-6 per group). 5 minutes after injection, 2 mm long sections of the tail, from the distil tip, were amputated. The amputated tail was immediately immersed in 14 ml of sterile saline at 37 C. The times until bleeding from the amputated tail stopped were recorded.

In Vivo Biodistribution.

3×10¹⁰ SynPlats, either plain or conjugated with CBP-VBP-RGD peptides, in saline were injected via tail vein into healthy female BALB/c mice (18-20 g; n=3-6 per group). 5 minutes following injection, 2 mm long sections of the tail, from the distil tip, were amputated. 55 minutes following tail amputation, animals were sacrificed via CO2 overdose and organs were collected. The organs were dissolved overnight in Solvable at a concentration of 100 mg organ per 1 ml of Solvable. Dissolved organ solutions were measured for their fluorescence at a concentration of 2 mg of organ per 200 ul Solvable. An identical amount of each organ from non-injected control animals were subtracted from each organ value for CBP-VBP-FMP SynPlats and plain SynPlat groups.

FTIR.

All FTIR samples were suspended in identical concentrations and volumes of a water/acetone mixture. Samples were pipetted onto a zinc selenide ATR crystal and water/acetone mixture was evaporated completely leaving a film of the sample. Samples were then placed into an FTIR spectrometer (NICOLET 4700, Thermo Electron Corporation) and the chamber was purged with nitrogen for 30 minutes. Dry crystal backgrounds were subtracted from each sample's spectrum.

Tail Sectioning.

The tail samples were harvested and then immediately frozen in OCT compound and sectioned at a thickness of 15 microns on a cryotome (Leica). After sectioning, the tail sections were mounted on a glass slide. 30 ul of Permount mounting medium (Fisher) were placed on top of the tail sections along with a glass cover slip to seal the slides.

Confocal Microscopy.

Individual imaging of SynPlats was performed on a Olympus Fluoview 1000 using differential interference contrast mode. A BX60 microscope was used to image tail sections.

### Results

Synthesis and Characterization of SynPlats.

Synthetic nanoplatelets (SynPlats) were synthesized using the layer-by-layer (LbL) approach, a proven method for creating a variety of flexible capsules that are mechanically and morphologically similar to circulatory cells. Briefly, 200 nm spherical polystyrene (PS) nanoparticles were coated with alternative layers of poly(allyamine) hydrochloride (PAH) and bovine serum albumin (BSA). PAH and BSA were chosen as the polycation and polyanion, respectively, due to their reliability in capsule synthesis via LbL as well as use as materials for numerous biomedical applications, for example, as described in del Mercato, L. L., et al., LbL multilayer capsules: recent progress and future outlook for their use in life sciences. Nanoscale, 2010. 2(4): p. 458-67; Johnston, A. P., et al., Layer-by-layer engineered capsules and their applications. Current Opinion in Colloid & Interface Science, 2006. 11(4): p. 203-209; and Yan, Y., M. Björnmalm, and F. Caruso, Assembly of Layer-by-Layer Particles and Their Interactions with Biological Systems. Chemistry of Materials, 2013.

SynPlats were characterized at each step for sufficient PAH/BSA coating via fluormetric assays. Briefly, PAH-AF594 and BSA-AF488 were complementarily coated and the fluorescent intensity for each dye was determined at each coating layer. 4 bi-layers were used in this example. The linear relationship of independently dyed polyelectrolytes implies uniform coating of both PAH and BSA. Coating was also confirmed qualitatively via confocal imaging of the final SynPlat product. PS core removal was performed via incubation with tetrahydrofuran (THF) and isopropyl alcohol (IPA) at increasing THF:IPA ratios. PS core removal was confirmed via FTIR. Since SynPlats are comprised of PS, PAH and BSA many similar peaks overlap and become difficult to resolve. However, wavenumbers 700 cm⁻¹ and 760 cm⁻¹ represent polystyrene peaks that are absent in both BSA and PAH. The resultant particles following removal of the core were oblate ellipsoidal in shape, which resembles natural platelets.

Peptide Conjugation to SynPlats.

The size of the particles (˜200 nm), however, was much smaller than that of natural platelets (˜2 mm). This was done in order to avoid cardiopulmonary interference. Specifically, particles larger than lung capillaries are known to physically get trapped in lungs (the first capillary bed encountered following tail vein injection) which can impede the passage of blood, effectively impairing oxygen delivery. Further, SEQ ID NO: 3 peptide has been shown to lead to lung targeting of hemostatic particles to lungs (over 50% of injected dose). These same SEQ ID NO: 3 particles, at high doses (40 mg/kg), have been shown to induce cardiopulmonary complications in animals; a finding substantiated by the fact that the parent protein of SEQ ID NO: 3, fibrinogen, also targets lung tissue in addition to inducing hemostasis. The combination of high lung targeting and physical entrapment due to large size can potentially lead to cardiopulmonary issues. To circumvent these issues, two steps were taken: (i) smaller (200 nm) templates were used so as to prevent physical entrapment in lung tissue and (ii) lower particle doses (15 mg/kg) were used.

These three peptides of the SynPlats, a CBP comprising SEQ ID NO: 1, a VBP comprising SEQ ID NO: 2, and a FMP comprising SEQ ID NO: 3 have been shown to act synergistically to promote hemostasis more efficiently, as described in Modery-Pawlowski, C. L., et al., In vitro and in vivo hemostatic capabilities of a functionally integrated platelet-mimetic liposomal nanoconstruct. Biomaterials, 2013. 34(12): p. 3031-41. Specifically, CBP and VBP promote adhesion at low and high shear, respectively, which are excluded from the overwhelming majority of platelet substitute designs. Conjugation of hemostatic peptides to SynPlats must be done in a way so as to ensure that peptides do not detach from SynPlats in vivo in order to avoid off-site activation of platelets. Further, due to the likelihood of peptides non-specifically interacting with the BSA rich surface on SynPlats and potentially unable to bind to target sites, peptides must be presented in a way so as to avoid direct interaction with the globular BSA-terminated layer of SynPlats to ensure high binding avidity and selectively. To accomplish this goal, branched dendrimers were used to first bridge the covalent attachment of the peptides to SynPlat surface. Briefly, the peptides were coupled to poly(amido amine) (PAMAM) dendrimers via EDC/NHS chemistry and the outer layer of SynPlats were activated with cabonyldiimidazole (CDI) where diaminoethane was then added to form primary amino groups on SynPlat surface. Dendrimer-peptide conjugates, were mixed and then directly conjugated to SynPlats via EDC/NHS chemistry. Peptide conjugation was quantified via fluorescent labeling of dendrimers and confirmed qualitatively via confocal microscopy. The number of peptide Molecules per SynPlat was (in ×10̂6 peptides/particle): CBP: 6.86+/−0.13; VBP: 7.08+/−0.18; and RGD: 14.7+/−0.15.

### Triggering of Hemostasis In Vivo Using SynPlats

SynPlats were next investigated in vivo for their ability to halt bleeding in a standard tail transection model in BALB/c mice. SynPlats without peptides and saline injections alone showed no decrease in tail bleeding times. SynPlats functionalized with SEQ ID NO: 3 peptide alone lowered bleeding time by ˜45%. However, SynPlats functionalized with all three peptides (SEQ ID NO: 1, SEQ ID NO: 2; and SEQ ID NO: 3) lowered bleeding time by ˜65%. Further, micron sized SynPlats were unable to instigate hemostasis to the same extent as their 200 nm counterparts, likely due to the lower circulation time of micron sized particles. Non-flexibile spherical 200 nm SynPlats with the PS core, identically decorated to their flexibile counterpart, were unable to cause hemostasis as rapidly as the more flexible, disc-shaped, SynPlats. Organ distribution for 200 nm SynPlats with and without peptide functionalization showed similar organ distribution except in case of the tail section containing the clot. In this case, a 3-fold increase in SynPlats functionalized with CBP-VBP-RGD peptides in the tail section containing the clot was seen over plain SynPlats.

The results demonstrated the ability of SynPlats to significantly reduce the bleeding time. In case of normal hemostatic plug formation, circulating platelets become activated and bind to the damaged endothelium due to exposure of collagen and release of vWF from wound site. In case of hemostatic plug formation following injection of SynPlats, activated circulating platelets and SynPlats both bind to injured endothelium, as well as to each other, effectively forming the hemostatic plug much faster than in the absence of SynPlats. Brightfield and fluorescent images show the interaction between fluorescently labeled SynPlats and the clot. On average, the hemostatic plug took around 195 seconds to form when it consisted of just natural circulating platelets. However, after an injection of SynPlats, the hemostatic plug formed in 35% of the time it took when no SynPlats were injected. The SynPlats described here offer a new tool for the treatment of serious hemorrhage.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1-49. (canceled)

50. A functionalized particle, comprising
a substrate for the attachment of dendrimer linkers;
dendrimer linkers, coupled to the surface of the substrate; and
one or more functional moieties at the terminal ends of the dendrimer linkers.

51. The functionalized particle of claim 50, wherein
the substrate comprises a shell comprising one or more bilayers, wherein each bilayer of the shell comprises a layer of cationic polyelectrolyte and a layer of anionic polyelectrolyte.

52. The functionalized particle of claim 51, wherein
each bilayer comprises a layer of BSA and a layer of poly (allylamine hydrochrolide).

53. The functionalized particle of claim 50, wherein
the dendrimer linkers comprise PAMAM dendrimers.

54. The functionalized particle of claim 50, wherein
the dendrimer linkers are omitted and the functional moieties are bound directly to the substrate.

55. The functionalized particle of claim 50, wherein
the one or more functional moieties comprise a wound targeting ligand and a platelet binding agent.

56. The functionalized particle of claim 55, wherein
the wound targeting ligand comprises SEQ ID NO: 1 or SEQ ID NO: 2 and
the platelet binding agent comprises SEQ ID NO: 3 or SEQ ID NO: 4.

57. The functionalized particle of claim 50, wherein
the functional moieties comprise a wound binding moiety and a wound healing peptide.

58. The functionalized particle of claim 57, wherein
the wound binding moiety comprises SEQ ID NO: 1 or SEQ ID NO: 2.

59. The functionalized particle of claim 50, wherein
the functional moieties comprise a wound or clot binding moiety and an anti-thrombotic agent.

60. The functionalized particle of claim 59, wherein
the wound or clot binding moiety comprises SEQ ID NO: 4 and the the anti-thrombotic agent is heparin.

61. The functionalized particle of claim 50, wherein
the functional moieties comprise a wound or clot binding moiety and a thrombolytic agent.

62. The functionalized particle of claim 61, wherein
the wound or clot binding moiety comprises SEQ ID NO: 4 and the thrombolytic agent is tPA.

63. The functionalized particle of claim 50, wherein
the one or more functional moieties comprise a wound or clot-binding moiety and a tPA-binding moiety.

64. The functionalized particle of claim 63, wherein
the wound or clot-binding moiety comprises SEQ ID NO: 4 and the tPA-binding moiety comprises SEQ ID NO: 5

65. A modified red blood cell, comprising
a red blood cell or fragment thereof, wherein one or more functional moieties having platelet activity are bonded to the surface of the red blood cell.

66. The modified red blood cell of claim 65, wherein
the one or more functional moieties comprises a collagen binding molecule.

67. The modified red blood cell of claim 66, wherein
the collagen binding moiety comprises SEQ ID NO: 1.

68. The modified red blood cell of claim 65, wherein
the one or more functional moieties comprises a von Willebrand factor binding agent.

69. The modified red blood cell of claim 68, wherein
the von Willebmd factor-binding agent comprises SEQ ID NO: 2.

70. The modified red blood cell of claim 65, wherein
the one or more functional moieties comprises a fibrinogen mimetic peptide.

71. The modified red blood cell of claim 70, wherein
the one or more fibrinogen mimetic peptide comprises SEQ ID NO: 3
